(19) United States
(12) Patent Application Publication
(10) Pub. No.: US 2009/0093047 A1
(43) Pub. Date: Apr. 9, 2009

(54) Kit and Device for Generating Chemiluminescence Radiation

(75) Inventors: Holger Klapproth, Freiburg (DE); Sonja Bender Mohry, Gundilfingen (DE)

Correspondence Address: THE WEBB LAW FIRM, P.C., 700 KOPPERS BUILDING, 436 SEVENTH AVENUE, PITTSBURGH, PA, 15219, US

(73) Assignee: MICRONAS HOLDING GMBH, Freiburg i.Br. (DE)

(21) Appl. No.: 12/182,676

(22) Filed: Jul. 30, 2008

(30) Foreign Application Priority Data
Aug. 3, 2007 (EP) 07 015 258.2

(51) Int. Cl. C12M 1/34

(52) U.S. Cl. 435/288.7

(57) ABSTRACT

A device for generating a chemiluminescence radiation depending on the binding of at least one ligand that is contained in a sample that is to be tested to at least one receptor that is binding-specific for the ligand has a flow cell having an internal cavity. The internal cavity has a separating wall on which the at least one receptor is immobilized. The internal cavity has at least two compartments. In a first compartment solid is deposited that contains at least one peroxidase-enzyme-possessing marker for marking the ligand in lyophilized form. A second compartment contains salt-stabilized luminol and a solid substance that releases hydrogen peroxide upon contact with water.

The invention relates to a kit for generating chemiluminescence radiation depending on the binding of at least one ligand that is contained in a sample that is to be tested to at least one receptor that is binding-specific for the ligand with a biochip that has a carrier on which the at least one receptor is immobilized, with at least one marker having at least one peroxidase enzyme for marking the ligand, and with luminol, which is stabilized with a salt. The invention also relates to a device for generating a chemiluminescence radiation depending on the binding of at least one ligand that is contained in a sample that is to be tested to at least one receptor that is binding-specific for the ligand with a flow cell having an internal cavity, and the internal cavity has a separating wall on which the at least one receptor is immobilized, with at least one marker having at least one peroxidase enzyme for marking the ligand and with luminol, which is stabilized with a salt.

A device of this type is known from DE 10 2004 050 032 A1. It has a measuring chamber in whose internal cavity receptors are immobilized on each of a plurality test sites arranged in the form of a matrix. The receptors are binding-specific for ligands that are assumed to be present in a sample that is to be tested. In order to test the sample, these ligands are brought into contact with the receptors in such a way that the ligands can bind to the receptors. Any free ligands that are still present are then removed from the measuring chamber. The ligands that are bound to a receptor are brought into contact with biotinylated detection antibodies, which bind to the ligands. The detection antibodies are then marked with a streptavidin peroxidase enzyme marker, and any free streptavidin peroxidase enzyme markers that may still be present are removed from the measuring chamber. The test sites are then brought into contact with hydrogen peroxide and luminol. When the peroxidase enzyme is present, the luminol decomposes, releasing a chemiluminescence radiation. This radiation is detected with the aid of sensors in order to detect the ligands and/or determine their concentration in the sample. The device has the disadvantage that the hydrogen peroxide is unstable and must be stored in a cooled condition. Furthermore, the peroxidase enzyme is very sensitive and is stored at −20° C. Therefore, the handling of the device and the required reagents is relatively complicated in actual practice. Furthermore, the device has limited suitability for decentralized use if a refrigerating apparatus is not present at the location where the device is being used.

DE 102 45 435 B4 also discloses a device that is provided for detecting ligands contained in a sample to be tested and that has a measuring chamber in which receptors are immobilized at each of a plurality of test sites. The detection of the binding of the ligands to the receptors is accomplished with the aid of detection antibodies that are marked with an optical marker and are deposited in lyophilized form in the measuring chamber. When the measuring chamber is filled with the sample, the lyophilized detection antibody marker complexes dissolve and bind to the ligands. Through exposure to an excitation radiation, the markers are excited so that they emit a luminescence radiation. This latter radiation is detected with the aid of optical sensors. The device may indeed be stored for a relatively long time period at room temperature without significant damage to the detection antibody marker complexes. However, the handling of the device remains relatively complicated because a light source is required in order to generate the excitation radiation for the markers.

Therefore, the object is to create a kit and a device of the type referred to above that permits simple handling.

This object is accomplished with respect to the kit of the type referred to above such that the kit has a solid substance that releases hydrogen peroxide upon contact with water, and such that the kit comprises a solid that contains the marker in lyophilized form.

The object referred to above is accomplished with respect to the device in such a way that the internal cavity has at least two compartments, that the luminol, which is stabilized with the salt, and a solid that contains the marker in lyophilized form are deposited in at least one first compartment, and at least one second compartment contains the luminol, which is stabilized with the at least one salt, and a solid substance, which releases hydrogen peroxide upon contact with water.

In an advantageous manner the kit or the device can then be stored at is room temperature for a relatively long time without significant changes in the properties of the substances required for the measurement. In this way a complicated cooling of the substances is not necessary. The kit is therefore particularly well suited for decentralized use, for example at the premises of a final consumer. As soon as the solid substance comes into contact with water, the hydrogen peroxide that is needed for generating the chemiluminescence radiation is released. Since the luminescence radiation is generated chemically, an excitation light source is not required for the measurement. Therefore the kit is easy to use.

In a preferred embodiment of the invention, the solid substance contains sodium peroxide (Na₂O₂). When the solid substance contacts water, it dissolves, causing an equilibrium to be established between the sodium peroxide and water on the one hand and sodium hydroxide and hydrogen peroxide on the other hand.

In another advantageous embodiment of the invention, the solid substance contains ammonium persulfate ((NH₄)₂S₂O₈). The hydrogen peroxide can also bind to this water-soluble substrate substance, so that it can be stored in solid form at room temperature.

The solid substance preferably contains urea. Such a solid substance is commercially available under the names Perhydrit® or Percarbamid®. It permits the hydrogen peroxide to be preserved at room temperature over a long time period in a manner that is largely insensitive to moisture and humidity.

The solid preferably contains a nonreducing sugar and a stabilized protein. The protein preferably comprises gelatin and/or a protein of the LEA class and/or a polypeptide of the LEA class and/or bovine serum albumin. The nonreducing sugar in this case is preferably trehalose. A method for preparing such a marker that has proven to be effective in actual practice is disclosed in EP 1 534 740 B1. It ensures that the biological activity of the peroxidase enzyme is achieved without cooling.

It is advantageous if the solid contains an antioxidant, preferably sodium ascorbate. In this case the peroxidase enzyme has a longer shelf life.

It is useful if the marker has at least one streptavidin peroxidase enzyme complex. If the streptavidin is dissolved in the sample, it can then bind to a detection antibody that is binding-specific for the ligand and in this way to indirectly mark the ligand with the peroxidase enzyme.

It is advantageous if the kit contains at least one detection antibody that is binding-specific for the ligand, or a functional fragment of such a detection antibody in lyophilized form. In the device of the invention such a lyophilized detection antibody or a functional fragment of such an antibody may be located in the at least one second compartment. The detection antibody may be configured in such a way that, upon contact with the peroxidase enzyme, it binds directly or indirectly to said enzyme. However, it is also possible for the detection antibody already to be bound to the peroxidase enzyme and for the corresponding detection antibody peroxidase enzyme complex to be present in lyophilized form.

In a preferred embodiment of the invention the internal cavity has a third compartment in which the at least one receptor is immobilized, and the first and the second compartment each have an inlet opening and each have an outlet opening that is connected to the third compartment. In this way it is possible to prevent the contact of the substances present in the first and second compartments with the at least one receptor located in the third compartment while the device is being stored.

The third compartment preferably has a fluid outlet, and the at least one receptor is located between the first and/or second compartment on the one hand and the fluid outlet on the other hand. In some cases an absorbent medium that absorbs the solution consisting of the sample and the substances stored in the first and second compartments following contact with the at least one receptor may be connected to the fluid outlet.

It should be noted that the salt-stabilized luminol and the solid substance may also be located in separate compartments.

An embodiment example of the invention is explained in greater detail below on the basis of the drawing. The drawing shows:

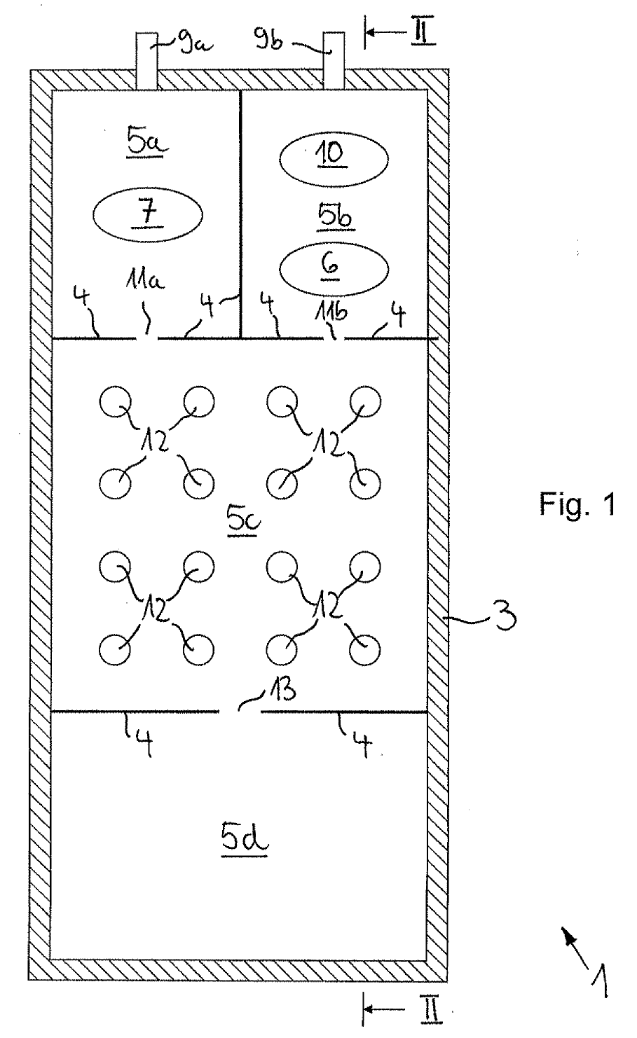

FIG. 1 a longitudinal sectional view through a device for generating a chemiluminescence radiation,

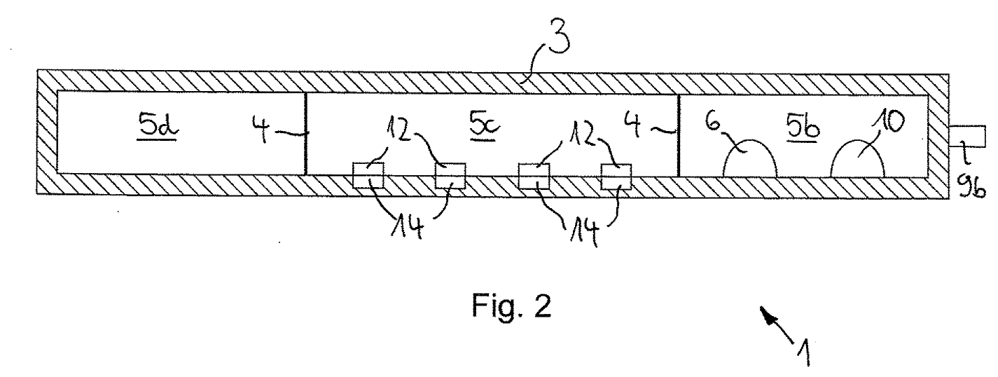

FIG. 2 a cross-section through the device along the plane designated II in FIG. 1, and

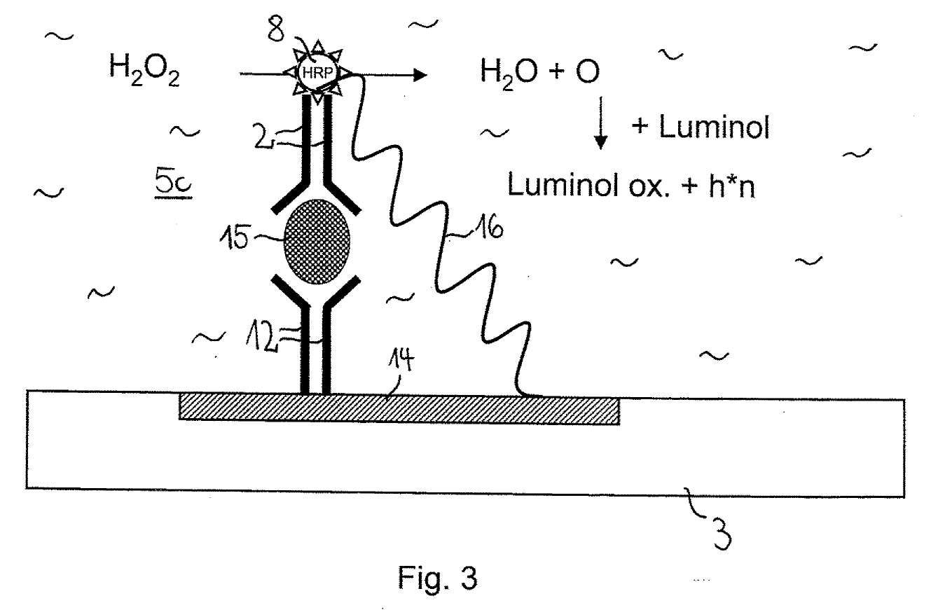

FIG. 3 a ligand bound by means of a detection antibody to a receptor and marled with an optical marker.

A device 1 for detecting and/or determining the concentration of ligands 2 in a water-containing liquid or free-flowing sample has a flow cell 3 with an internal cavity that is divided by dividing walls 4 into a plurality of compartments 5a, 5b, 5c, 5d.

In a first compartment 5a a solid 7 is deposited that contains streptavidin-HRP complexes in lyophilized form as optical markers 8. In order to stabilize the markers 8, the solid 7 contains a nonreducing sugar, a protein of the LEA class, and an antioxidant.

The first compartment 5a has a first inlet opening 9a through which the sample can be filled, for example by means of a pipette, into the first compartment 5a. Upon contact with the sample, the solid 7 in the sample dissolves.

And a second compartment 5b salt-stabilized luminol 6 and a solid substance 10 are deposited, preferably at a distance from each other. The solid substance 10 contains a carrier, in particular urea, to which hydrogen peroxide is bound. The second compartment 5b has a second inlet opening 9b through which the sample and/or an aqueous liquid can be filled into the second compartment 5b.

Upon contact with the sample and/or the aqueous liquid, the luminol and the urea dissolve in the liquid, whereby the hydrogen peroxide is released. The first compartment 5a and the second compartment 5b each have an outlet opening 11a, 11b that is connected to a third compartment 5c. Valves that prevent the liquid that is located in the third compartment 5c from flowing back into the first compartment 5a and/or into the second compartment 5c may be located at the output openings 11a, 11b. As can be seen particularly well in FIG. 1, the luminol 6 and the solid 7 are deposited between the first inlet opening 9a and the outlet opening 11a. In a corresponding manner, the solid substance 10 is located between the second inlet opening 9b and the outlet opening 11b.

In a third compartment 5c a plurality of test sites are provided in the form of a matrix on a separating wall of the internal cavity. At least one $ receptor 12 that is binding-specific for a ligand 2 and that is suspected of being contained in the sample is immobilized at each test site. At least one optical sensor 14 is integrated into the separating wall of the flow cell 3 under each test site.

The third compartment 5c has a fluid outlet 13 that is connected to a fourth compartment 5d. In the fourth compartment 5d an absorbent medium, which is not shown in the drawing, is located. The receptors 12 are located between the output openings 11a and 11b and the fluid outlet 13. In order to test the sample, the sample is first filled through the first inlet opening 9a into the first compartment 5a. When this is done the solid 7 in the sample dissolves and the solution passes through the outlet opening 11a into the third compartment 5c.

In the third compartment 5c the sample comes in contact with the receptors 12 in such a way that the ligands 2—should they be contained in the sample—can bind to the receptors 12. Detection antibodies 15 that are bound and/or bind to the ligands 2 are contained in the sample. A rinsing liquid is now introduced through the first inlet opening 9a and the outlet opening 11a into the third compartment 5c and removes any free ligands 2 and/or detection antibodies 15 that may still be present from the third compartment 5c and transports them into the fourth compartment 5d.

The sample or the aqueous liquid referred to above is then filled through the second inlet opening 9b into the second compartment 5b in order to release the luminol 6 and the solid substance 6. This releases hydrogen peroxide, which, together with the luminol 6, passes through the outlet opening 11b into the third compartment 5c.

When the hydrogen peroxide and the luminol come into contact with a streptavidin-HRP complex that is bound to a receptor 12, the luminol breaks down, releasing chemiluminescence radiation 16 (FIG. 3). The chemiluminescence radiation 16 is detected at the individual test sites, in each case with the aid of the sensor 14. Depending on the measurement signals that are obtained in this way, the concentration of the respective ligands 2 in the sample is determined.

1. A kit for generating chemiluminescence radiation depending on the binding of at least one ligand that is contained in a sample that is to be tested to at least one receptor that is binding-specific for the ligand with a biochip that has a carrier on which the at least one receptor is immobilized, with at least one marker having at least one peroxidase enzyme for marking the ligand, and with luminol, which is stabilized with a salt, characterized in that the kit has a solid substance that upon contact with water releases hydrogen peroxide, and in that the kit comprises a solid that contains the marker in lyophilized form.

2. A device for generating chemiluminescence radiation depending on the binding of at least one ligand that is contained in a sample that is to be tested to at least one receptor that is binding-specific for the ligand with a flow cell having an internal cavity, and the internal cavity has a separating wall on which the at least one receptor is immobilized, with at least one marker having at least one peroxidase enzyme for marking the ligand and with luminol, which is stabilized with at least one salt, characterized in that the internal cavity has at least two compartments in that a solid that contains the marker in lyophilized form is deposited in at least one first compartment, and in that the luminol, which is stabilized with the salt, and a solid substance, which releases hydrogen peroxide upon contact with water are contained in at least one second compartment.

3. The kit of claim 1, wherein the solid substance contains sodium peroxide.

4. The kit of claim 1, wherein the solid substance contains ammonium persulfate.

5. The kit of claim 1, wherein the solid substance contains urea.

6. The kit of claim 1, wherein the solid substance contains a nonreducing sugar and a stabilized protein.

7. The kit of claim 1, wherein the stabilized protein contains gelatin and/or a protein of the LEA class and/or a polypeptide of the LEA class and/or bovine serum albumin.

8. The kit of of claim 1, wherein the solid substance contains an antioxidant, preferably sodium ascorbate.

9. The kit of claim 1, wherein the kit contains at least one detection antibody that is binding-specific for the ligand, or contains a functional fragment of said antibody in lyophilized for, and the detection antibody is bound to the peroxidase enzyme, and/or upon contact with the peroxidase enzyme is directly or indirectly formed onto said peroxidase enzyme.

10. The kit of claim 1, wherein the marker has at least one streptavidin-peroxidase-enzyme complex.

11. The device of claim 2, wherein the at least one first compartment contains at least one detection antibody that is binding-specific for the ligand, or contains a functional fragment of said antibody in lyophilized form.

12. The device of claim 2, wherein the internal cavity has a third compartment in which the at least one receptor is immobilized, and the first compartment and the second compartment each have an inlet opening and each have an outlet opening that is connected to the third compartment.

13. The device of claim 2, wherein the third compartment has a liquid outlet, and the at least one receptor is located between the first compartment and/or second compartment on the one hand and the fluid outlet on the other hand.

14. The device of claim 2, wherein the solid substance contains one of the group consisting of sodium peroxide, ammonium persulfate, urea, nonreducing sugar, stabilized protein, gelatin, protein of the LEA class, polypeptide of the LEA class, bovine serum albumin and sodium ascorbate.

15. The device of claim 2, wherein the device contains at least one detection antibody that is binding-specific for the ligand, or contains a functional fragment of said antibody in lyophilized form, and the detection antibody is bound to the peroxidase enzyme, and/or upon contact with the peroxidase enzyme is directly or indirectly formed onto said peroxidase enzyme.

16. The device of claim 2, wherein the marker has at least one streptavidin-peroxidase-enzyme complex.
